(19) United States
(12) Patent Application Publication
(10) Pub. No.: US 2010/0311798 A1
(43) Pub. Date: Dec. 9, 2010

(54) 2-AMINOOXAZOLINES AS TAAR1 LIGANDS

(76) Inventors: Guillaume Décoret, Schlierbach (FR); Guido Galley, Rheinfelden (DE); Katrin Groebke Zbinden, Liestal (CH); Roger Norcross, Olsberg (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 12/789,484

(22) Filed: May 28, 2010

(30) Foreign Application Priority Data
Jun. 5, 2009 (EP) 09162061.7

(51) Int. Cl. A61K 31/421; C07D 263/28

(52) U.S. Cl. 514/377; 548/233

(57) ABSTRACT

The invention relates to compounds of formula

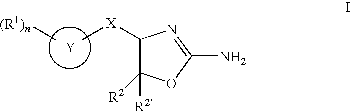

wherein
R¹, R², R^{2′}, X, Y, andn are as defined in the specification or to a pharmaceutically suitable acid addition salt thereof. The compounds of formula I are active on the TAAR1 receptor and are therefore suitable for the treatment of depression, anxiety disorders, bipolar disorder, attention deficit hyperactivity disorder, stress-related disorders, psychotic disorders, schizophrenia, neurological diseases, Parkinson's disease, neurodegenerative disorders, Alzheimer's disease, epilepsy, migraine, substance abuse and metabolic disorders, eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders.

### PRIORITY TO RELATED APPLICATION(S)

This application claims the benefit of European Patent Application No. 09162061.7, filed Jun. 5, 2009, which is hereby incorporated by reference in its entirety.

### BACKGROUND OF THE INVENTION

2-Aminooxazolines are described in the literature as hypertensive agents with good affinity to the adrenergic receptor or as intermediates in processes for preparation of pharmaceutical active agents, for example in EP 0 167 459, U.S. Pat. No. 4,311,840, DE 2,253, 555, Tetrahedron (2001), 57(1), 195-200 or in Bioorganic and Medicinal Chemistry Letters (2004), 14(2), 313-316.

Some of the physiological effects (i.e. cardiovascular effects, hypotension, induction of sedation) which have been reported for compounds which may bind to adrenergic receptors (WO02/076950, WO97/12874 or EP 0717 037) may be considered to be undesirable side effects in the case of medicaments aimed at treating diseases of the central nervous system as described above. Therefore it is desirable to obtain medicaments having selectivity for the TAAR1 receptor vs adrenergic receptors.

The classical biogenic amines (serotonin, norepinephrine, epinephrine, dopamine, histamine) play important roles as neurotransmitters in the central and peripheral nervous system [Deutch, A. Y. and Roth, R. H. (1999) Neurotransmitters. In Fundamental Neuroscience (2^{nd} edn) (Zigmond, M. J., Bloom, F. E., Landis, S. C., Roberts, J. L, and Squire, L. R., eds.), pp. 193-234, Academic Press]. Their synthesis and storage, as well as their degradation and reuptake after release are tightly regulated. An imbalance in the levels of biogenic amines is known to be responsible for the altered brain function under many pathological conditions [Wong, M. L. and Licinio, J. (2001) Research and treatment approaches to depression. Nat. Rev. Neurosci. 2, 343-351; Carlsson, A. et al. (2001) Interactions between monoamines, glutamate, and GABA in schizophrenia: new evidence. Annu. Rev. Pharmacol. Toxicol. 41, 237-260; Tuite, P. and Riss, J. (2003) Recent developments in the pharmacological treatment of Parkinson's disease. Expert Opin. Investig. Drugs 12, 1335-1352, Castellanos, F. X. and Tannock, R. (2002) Neuroscience of attention-deficit/hyperactivity disorder: the search for endophenotypes. Nat. Rev. Neurosci. 3, 617-628]. A second class of endogenous amine compounds, the so-called trace amines (TAs) significantly overlap with the classical biogenic amines regarding structure, metabolism and subcellular localization. The TAs include p-tyramine, β-phenylethylamine, tryptamine and octopamine, and they are present in the mammalian nervous system at generally lower levels than classical biogenic amines [Usdin, Earl; Sandler, Merton; Editors. Psychopharmacology Series, Vol. 1: Trace Amines and the Brain. [Proceedings of a Study Group at the 14th Annual Meeting of the American College of Neuropsychoparmacology, San Juan, Puerto Rico] (1976)].

Their dysregulation has been linked to various psychiatric diseases like schizophrenia and depression [Lindemann, L. and Hoener, M. (2005) A renaissance in trace amines inspired by a novel GPCR family. Trends in Pharmacol. Sci. 26, 274-281] and for other conditions like attention deficit hyperactivity disorder, migraine headache, Parkinson's disease, substance abuse and eating disorders [Branchek, T. A. and Blackburn, T. P. (2003) Trace amine receptors as targets for novel therapeutics: legend, myth and fact. Curr. Opin. Pharmacol. 3, 90-97; Premont, R. T. et al. (2001) Following the trace of elusive amines. Proc. Natl. Acad. Sci. U.S.A. 98, 9474-9475].

For a long time, TA-specific receptors had only been hypothesized based on anatomically discrete high-affinity TA binding sites in the CNS of humans and other mammals [Mousseau, D. D. and Butterworth, R. F. (1995) A high-affinity [3H] tryptamine binding site in human brain. Prog. Brain Res. 106, 285-291; McCormack, J. K. et al. (1986) Autoradiographic localization of tryptamine binding sites in the rat and dog central nervous system. J. Neurosci. 6, 94-101]. Accordingly, the pharmacological effects of TAs were believed to be mediated through the well known machinery of classical biogenic amines, by either triggering their release, inhibiting their reuptake or by “crossreacting” with their receptor systems [Premont, R. T. et al. (2001) Following the trace of elusive amines. Proc. Natl. Acad. Sci. U.S.A. 98, 9474-9475; Dyck, L. E. (1989) Release of some endogenous trace amines from rat striatal slices in the presence and absence of a monoamine oxidase inhibitor. Life Sci. 44, 1149-1156; Parker, E. M. and Cubeddu, L. X. (1988) Comparative effects of amphetamine, phenylethylamine and related drugs on dopamine efflux, dopamine uptake and mazindol binding. J. Pharmacol. Exp. Ther. 245, 199-210]. This view changed significantly with the recent identification of several members of a novel family of GPCRs, the trace amine associated receptors (TAARs) [Lindemann, L. and Hoener, M. (2005) A renaissance in trace amines inspired by a novel GPCR family. Trends in Pharmacol. Sci. 26, 274-281; Lindemann, L. et al. (2005) Trace amine associated receptors form structurally and functionally distinct subfamilies of novel G protein-coupled receptors. Genomics 85, 372-385]. There are 9 TAAR genes in human (including 3 pseudogenes) and 16 genes in mouse (including 1 pseudogene). The TAAR genes do not contain introns (with one exception, TAAR2 contains 1 intron) and are located next to each other on the same chromosomal segment. The phylogenetic relationship of the receptor genes, in agreement with an in-depth GPCR pharmacophore similarity comparison and pharmacological data suggest that these receptors form three distinct subfamilies [Lindemann, L. and Hoener, M. (2005) A renaissance in trace amines inspired by a novel GPCR family. Trends in Pharmacol. Sci. 26, 274-281; Lindemann, L. et al. (2005) Trace amine associated receptors form structurally and functionally distinct subfamilies of novel G protein-coupled receptors. Genomics 85, 372-385]. TAAR1 is in the first subclass of four genes (TAAR1-4) highly conserved between human and rodents. TAs activate TAAR1 via Gαs. Dysregulation of TAs was shown to contribute to the aetiology of various diseases like depression, psychosis, attention deficit hyperactivity disorder, substance abuse, Parkinson's disease, migraine headache, eating disorders, metabolic disorders and therefore TAAR1 ligands have a high potential for the treatment of these diseases.

Therefore, there is a broad interest to increase the knowledge about trace amine associated receptors.

### SUMMARY OF THE INVENTION

The invention provides compounds of formula I

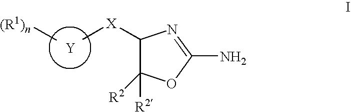

wherein
R¹ is halogen;
R² is lower alkyl or lower alkyl substituted by halogen;
R^{2′} is hydrogen, lower alkyl or lower alkyl substituted by halogen;
X is a bond, —CH₂—, —CH₂CH₂— or —CH₂CH₂CH₂—;
Y is phenyl or cyclohexyl; and
n is 0, 1 or 2;
or a pharmaceutically suitable acid addition salt thereof.

The invention includes all racemic mixtures, all their corresponding enantiomers and/or optical isomers. In addition, all tautomeric forms of compounds of formula I are also encompassed by the present invention.

The invention also provides pharmaceutical compositions which comprise a compound of formula I and a pharmaceutically acceptable carrier. The invention further provides methods for the manufacture of the compounds and compositions for the invention.

Compounds of formula I have a good affinity to the trace amine associated receptors (TAARs), especially for TAAR1 receptor over adrenergic receptors, in particular good selectivity vs the human and rat alpha1 and alpha2 adrenergic receptors.

The compounds can be used for the treatment of depression, anxiety disorders, bipolar disorder, attention deficit hyperactivity disorder (ADHD), stress-related disorders, psychotic disorders such as schizophrenia, neurological diseases such as Parkinson's disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders. Thus, the invention also provides such methods.

The preferred indications using the compounds of the present invention are depression, psychosis, Parkinson's disease, diabetes, anxiety and attention deficit hyperactivity disorder (ADHD).

### DETAILED DESCRIPTION OF THE INVENTION

As used herein, the term “lower alkyl” denotes a saturated straight- or branched-chain group containing from 1 to 7 carbon atoms, for example, methyl, ethyl, propyl, isopropyl, n-butyl, i-butyl, 2-butyl, t-butyl and the like. Preferred alkyl groups are groups with 1-4 carbon atoms.

As used herein, the term “lower alkyl substituted by halogen” denotes an alkyl group as defined above, wherein at least one hydrogen atom is replaced by halogen, for example CF₃, CHF₂, CH₂F, CH₂CF₃, CH₂CH₂CF₃, CH₂CF₂CF₃ and the like.

The term “halogen” denotes chlorine, iodine, fluorine and bromine.

“Pharmaceutically acceptable,” such as pharmaceutically acceptable carrier, excipient, etc., means pharmacologically acceptable and substantially non-toxic to the subject to which the particular compound is administered.

The term “pharmaceutically acceptable acid addition salts” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methane-sulfonic acid, p-toluenesulfonic acid and the like.

“Therapeutically effective amount” means an amount that is effective to prevent, alleviate or ameliorate symptoms of disease or prolong the survival of the subject being treated.

In one embodiment, the invention provides compounds of formula I having one of the following formulas

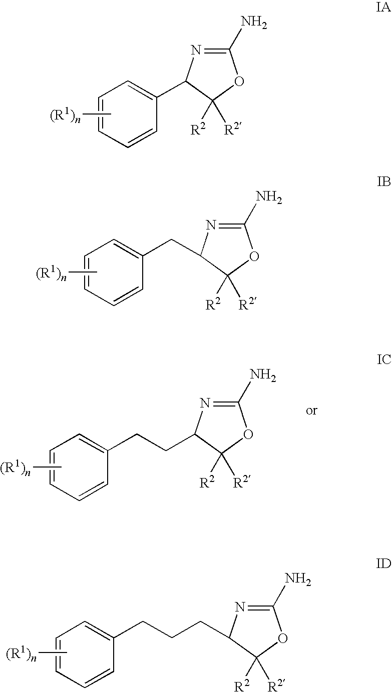

wherein
R¹ is halogen;
R² is lower alkyl or lower alkyl substituted by halogen;
R²′ is hydrogen, lower alkyl or lower alkyl substituted by halogen; and
n is 0, 1 or 2.

In particular, the invention provides the following specific compounds:
(4R,5S)-5-methyl-4-phenethyl-4,5-dihydro-oxazol-2-ylamine;
(4S,5S)-5-methyl-4-phenethyl-4,5-dihydro-oxazol-2-ylamine;
(4 S,5R)-5-methyl-4-phenethyl-4,5-dihydro-oxazol-2-ylamine;
(4 S)-5-ethyl-4-phenethyl-4,5-dihydro-oxazol-2-ylamine;
(4S,5R)-5-methyl-4-(3-phenyl-propyl)-4,5-dihydro-oxazol-2-ylamine;
(4S,5S)-5-methyl-4-(3-phenyl-propyl)-4,5-dihydro-oxazol-2-ylamine;
(4S,5S)-4-benzyl-5-methyl-4,5-dihydro-oxazol-2-ylamine;
(4S,5R)-4-benzyl-5-methyl-4,5-dihydro-oxazol-2-ylamine;
(4S,5R)-4-[2-(4-chloro-phenyl)-ethyl]-5-methyl-4,5-dihydro-oxazol-2-ylamine;
(4S,5S)-4-[2-(4-chloro-phenyl)-ethyl]-5-methyl-4,5-dihydro-oxazol-2-ylamine;
(4S,5R)-4-[2-(3,4-dichloro-phenyl)-ethyl]-5-methyl-4,5-dihydro-oxazol-2-ylamine;
(4S,5S)-4-[2-(3,4-dichloro-phenyl)-ethyl]-5-methyl-4,5-dihydro-oxazol-2-ylamine; and
(4S,5R)-4-[3-(4-chloro-phenyl)-propyl]-5-methyl-4,5-dihydro-oxazol-2-ylamine.

In another embodiment, the invention provides compounds of formula I having one of the following formulas

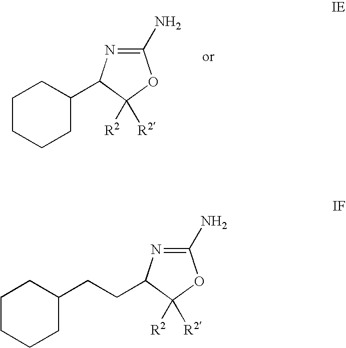

wherein
R² is lower alkyl or lower alkyl substituted by halogen;
R^{2′} is hydrogen, lower alkyl or lower alkyl substituted by halogen; and
n is 1 or 2.

In particular, the invention provides the following compounds:
(4-cyclohexyl-5-methyl-4,5-dihydro-oxazol-2-ylamine;
(4S,5S)-4-(2-cyclohexyl-ethyl)-5-methyl-4,5-dihydro-oxazol-2-ylamine; and
(4S,5R)-4-(2-cyclohexyl-ethyl)-5-methyl-4,5-dihydro-oxazol-2-ylamine.

The compounds of formula I and their pharmaceutically acceptable salts can be prepared by methods known in the art, for example, by processes described below, which process comprises

a) Reacting a compound of formula

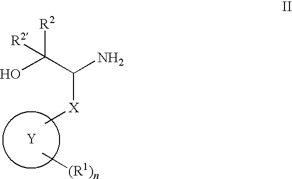

with cyanogen bromide
to obtain a compound of formula

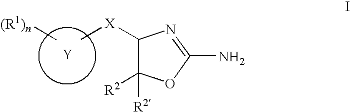

wherein the definitions are as described above, or

if desired, converting the compounds obtained into pharmaceutically acceptable acid addition salts.

The preparation of compounds of formula I of the present invention can be carried out in sequential or convergent synthetic routes. Syntheses of the compounds of the invention are shown in the following schemes 1-5. The skills required for carrying out the reaction and purification of the resulting products are known to those skilled in the art. The substituents and indices used in the following description of the processes have the significance given herein before unless indicated to the contrary.

In more detail, the compounds of formula I can be manufactured by the methods given below, by the methods given in the examples or by analogous methods. Appropriate reaction conditions for the individual reaction steps are known to a person skilled in the art. The reaction sequence is not limited to the one displayed in schemes 1 to 5, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

### General Procedure

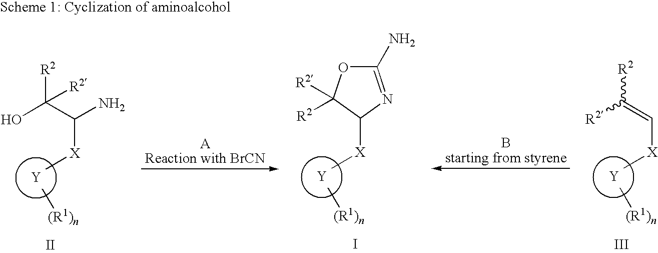

Step A: Cyclization of the amino alcohol II to the corresponding 2-aminooxazoline I can be accomplished by treatment with cyanogen bromide in THF as solvent and K₂CO₃ as base at r.t. overnight, or by treatment with cyanogen bromide in methanol as solvent and sodium acetate as base at 0° C. to r.t. overnight.

Step B: Amino-oxazoline ring formation can be accomplished by a two-step procedure comprising treatment of a corresponding alkene with silver cyanate and iodine in a solvent mixture such as ethyl acetate/acetonitrile at temperatures from 0° C. to room temperature for 1-18 hrs, followed by reaction with aqueous ammonia at room temperature.

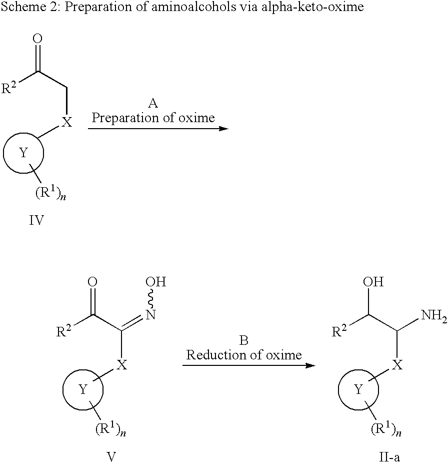

Step A: Oxime formation V is effected either by treatment of the ketone IV with sodium nitrite in AcOH or tert-butyl nitrite in EtOH in the presence of NaOEt.

Step B: Reduction of the oxime V to the aminoalcohol II-a is effected either by hydrogenation at elevated pressure (130 bar) in the presence of Raney nickel as catalyst (leaving an aromatic ring Y intact) or by hydrogenation at elevated pressure (2.5 bar) in the presence of PtO₂ as catalyst (leading to saturation of an aromatic ring Y).

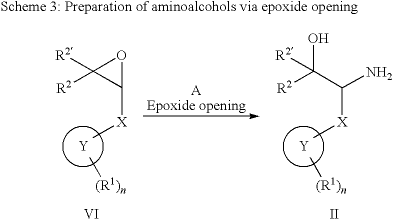

Step A: Conversion of epoxide VI to aminoalcohol II is effected by treatment with 25% aqueous NH₃-solution in the presence of lithium perchlorate at 125° C. (autoclave).

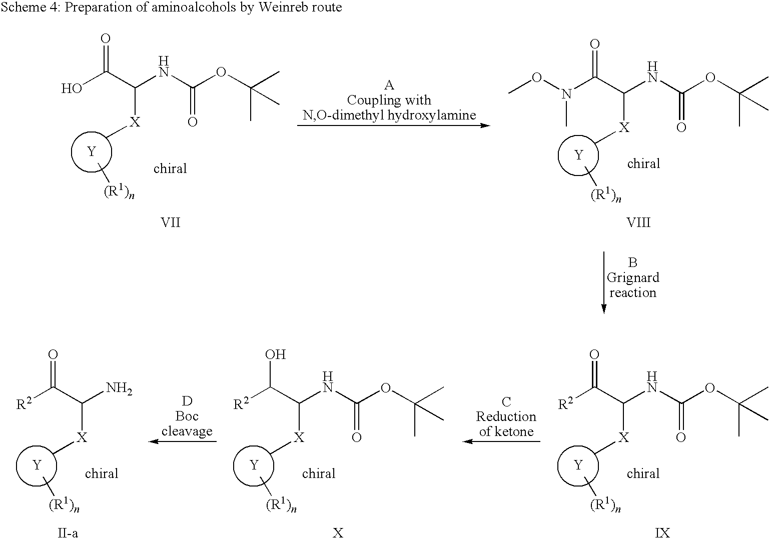

Step A: The Weinreb amide VIII is prepared by coupling acid VII with N,O-dimethyl hydroxylamine in a suitable solvent such as CH₂Cl₂, DMF, acetonitrile, THF using activation by an amide coupling reagent such as BOP, BOP-Cl, TBTU, EDCI, EDCI/DMAP in the presence of a base like TEA, DIPEA, N-methylmorpholine etc. at 0° C. to 50° C. Reaction times range from 1 hr-72 hrs.

Preferred conditions are CH₂Cl₂, EDCI and N-methyl morpholine at 0° C. for 4 hrs.

Step B: Conversion of the Weinreb amide VIII to the corresponding alkyl ketone IX is accomplished by treatment with an alkyl Grignard reagent in a solvent such as THF, diethylether at −40° C. −40° C. for 1-8 hrs. Preferred conditions are methyl magnesium chloride in THF at r.t. for 1.5 hr.

Step C: Reduction of a ketone IX is achieved with a reductant such NaBH₄, LiBH₄, DIBAH, LiAlH₄, BH₃ or BH₃-dimethylsulfide in a solvent such as MeOH, EtOH, THF, diethylether or toluene at −78° C. −50° C. for 1-24 hrs.

Preferred conditions are NaBH₄ in EtOH at r.t. overnight. A mixture of epimers is formed.

Step D: Cleavage of the amino protecting group can be effected with a variety of methods known in the art. The tert-butoxycarbonyl group can be cleaved using a mineral acid such as HCl, H₂SO₄ or H₃PO₄ or a organic acid such as CF₃COOH, CHCl₂COOH, HOAc or p-toluonesulfonic acid in a solvent such as CH₂Cl₂, CHCl₃, THF, MeOH, EtOH or H₂O at 0 to 60° C.

Preferred conditions are CF₃COOH in dichloromethane at room temperature overnight.

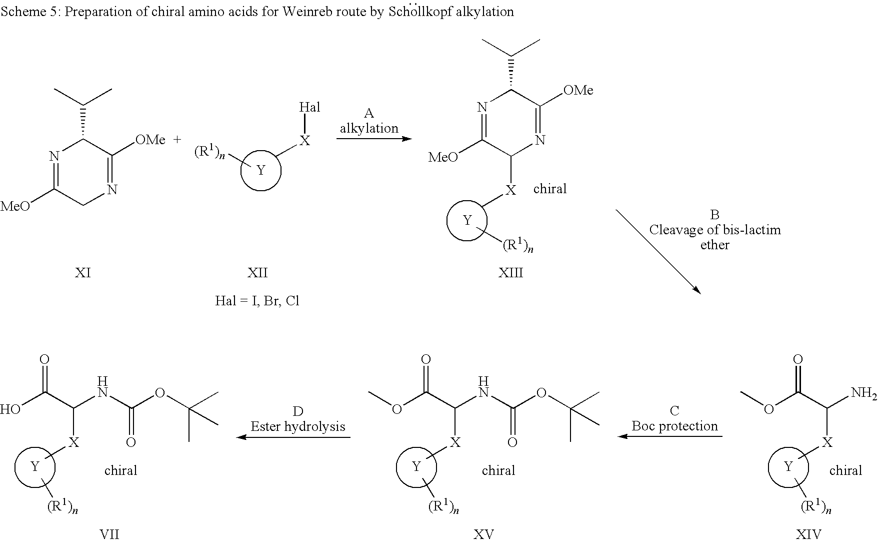

Step A: Deprotonation of bis-lactimether XI (also called “Schöllkopf's chiral auxiliary”) with a suitable base such as n-butyl-lithium, tert-butyl-lithium or LiHMDS in an appropriate organic solvent such as tetrahydrofuran, optionally in the presence of an auxiliary such as EDTA, TMEDA, DMI or HMPA at a low temperature followed by addition of the haloalkane XII and reaction for several hours leads to product XIII (Vassiliou, S. et at Synlett 2003, 2398-2400; Schöllkopf, U. Topics Curr. Chem. 1983, 109, 65).

Preferred conditions are the use of 1.6M n-butyl lithium solution as base with HMPA as additive in THF as solvent at −78° C. and allowing the mixture to reach room temperature overnight.

Step B: Bis-lactim ether product XIII is cleaved under acidic conditions using a mineral acid such as HCl, H₂SO₄ or H₃PO₄ or an organic acid such as CF₃COOH, CHCl₂COOH, HOAc or p-toluonesulfonic acid in a solvent such as acetonitrile, CH₂Cl₂, CHCl₃, THF, MeOH, EtOH or H₂O at 0 to 60° C.

Preferred conditions are a trifluoroacetic acid in a mixture of water and acetonitrile (1:3 to 6:1) r.t. overnight.

Step C: Boc protection of amino ester XIV is accomplished by treatment with Boc anhydride in a suitable solvent such as acetonitrile, CH₂Cl₂, EtOAc, dioxane, MeOH or THF in the presence of a base such as triethylamine, DIPEA, pyridine, Na₂CO₃, NaHCO₃.

Step D: Hydrolysis of ester XV is effected by dissolving it in a suitable solvent like MeOH, EtOH, THF, 1,4-dioxane, water or mixtures thereof and a base like LiOH, NaOH, KOH, Na₂CO₃, K₂CO₃ or Cs₂CO₃. Preferred conditions are NaOH in EtOH/H₂O.

### Isolation and Purification of the Compounds

Isolation and purification of the compounds and intermediates described herein can be effected, if desired, by any suitable separation or purification procedure such as, for example, filtration, extraction, crystallization, column chromatography, thin-layer chromatography, thick-layer chromatography, preparative low or high-pressure liquid chromatography or a combination of these procedures. Specific illustrations of suitable separation and isolation procedures can be had by reference to the preparations and examples herein below. However, other equivalent separation or isolation procedures could, of course, also be used. Racemic mixtures of chiral compounds of formula I can be separated using chiral HPLC.

### Salts of Compounds of Formula I

The compounds of formula I are basic and can be converted to a corresponding acid addition salt. The conversion is accomplished by treatment with at least a stoichiometric amount of an appropriate acid, such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Typically, the free base is dissolved in an inert organic solvent such as diethyl ether, ethyl acetate, chloroform, ethanol or methanol and the like, and the acid added in a similar solvent. The temperature is maintained between 0° C. and 50° C. The resulting salt precipitates spontaneously or can be brought out of solution with a less polar solvent.

The acid addition salts of the basic compounds of formula I can be converted to the corresponding free bases by treatment with at least a stoichiometric equivalent of a suitable base such as sodium or potassium hydroxide, potassium carbonate, sodium bicarbonate, ammonia, and the like.

The compounds of formula I and their pharmaceutically usable addition salts possess valuable pharmacological properties. Compounds of the present invention have a good affinity to the trace amine associated receptors (TAARs), especially TAAR1.

The compounds were investigated in accordance with the test given hereinafter.

### Materials and Methods

### Construction of TAAR Expression Plasmids and Stably Transfected Cell Lines

For the construction of expression plasmids the coding sequences of human, rat and mouse TAAR 1 were amplified from genomic DNA essentially as described by Lindemann et al. [14]. The Expand High Fidelity PCR System (Roche Diagnostics) was used with 1.5 mM Mg²⁺ and purified PCR products were cloned into pCR2.1-TOPO cloning vector (Invitrogen) following the instructions of the manufacturer. PCR products were subcloned into the pIRESneo2 vector (BD Clontech, Palo Alto, Calif.), and expression vectors were sequence verified before introduction in cell lines.

HEK293 cells (ATCC # CRL-1573) were cultured essentially as described Lindemann et al. (2005). For the generation of stably transfected cell lines HEK293 cells were transfected with the pIRESneo2 expression plasmids containing the TAAR coding sequences (described above) with Lipofectamine 2000 (Invitrogen) according to the instructions of the manufacturer, and 24 hrs post transfection the culture medium was supplemented with 1 mg/ml G418 (Sigma, Buchs, Switzerland). After a culture period of about 10 d clones were isolated, expanded and tested for responsiveness to trace amines (all compounds purchased from Sigma) with the cAMP Biotrak Enzyme immunoassay (EIA) System (Amersham) following the non-acetylation EIA procedure provided by the manufacturer. Monoclonal cell lines which displayed a stable EC₅₀ for a culture period of 15 passages were used for all subsequent studies.

### Membrane Preparation and Radioligand Binding

Cells at confluence were rinsed with ice-cold phosphate buffered saline without Ca²⁺ and Mg²⁺ containing 10 mM EDTA and pelleted by centrifugation at 1000 rpm for 5 min at 4° C. The pellet was then washed twice with ice-cold phosphate buffered saline and cell pellet was frozen immediately by immersion in liquid nitrogen and stored until use at −80° C. Cell pellet was then suspended in 20 ml HEPES-NaOH (20 mM), pH 7.4 containing 10 mM EDTA, and homogenized with a Polytron (PT 3000, Kinematica) at 10,000 rpm for 10 s. The homogenate was centrifuged at 48,000×g for 30 min at 4° C. and the pellet resuspended in 20 ml HEPES-NaOH (20 mM), pH 7.4 containing 0.1 mM EDTA (buffer A), and homogenized with a Polytron at 10,000 rpm for 10 s. The homogenate was then centrifuged at 48,000×g for 30 min at 4° C. and the pellet resuspended in 20 ml buffer A, and homogenized with a Polytron at 10,000 rpm for 10 s. Protein concentration was determined by the method of Pierce (Rockford, Ill.). The homogenate was then centrifuged at 48,000×g for 10 min at 4° C., resuspended in HEPES-NaOH (20 mM), pH 7.0 including MgCl₂ (10 mM) and CaCl₂ (2 mM) (buffer B) at 50 ug protein per ml and homogenized with a Polytron at 10,000 rpm for 10 seconds.

Binding assay was performed at 4° C. in a final volume of 1 ml, and with an incubation time of 30 min. The radioligand [³H]-rac-2-(1,2,3,4-tetrahydro-1-naphthyl)-2-imidazoline was used at a concentration equal to the calculated K_{d} value of 60 nM to give a bound at around 0.1% of the total added radioligand concentration, and a specific binding which represented approximately 70-80% of the total binding. Non-specific binding was defined as the amount of [³H]-rac-2-(1,2,3,4-tetrahydro-1-naphthyl)-2-imidazoline bound in the presence of the appropriate unlabelled ligand (10 μM). Competing ligands were tested in a wide range of concentrations (10 pM-30 μM). The final dimethylsulphoxide concentration in the assay was 2%, and it did not affect radioligand binding. Each experiment was performed in duplicate. All incubations were terminated by rapid filtration through UniFilter-96 plates (Packard Instrument Company) and glass filter GF/C, pre-soaked for at least 2 h in polyethylenimine 0.3%, and using a Filtermate 96 Cell Harvester (Packard Instrument Company). The tubes and filters were then washed 3 times with 1 ml aliquots of cold buffer B. Filters were not dried and soaked in Ultima gold (45 μl/well, Packard Instrument Company) and bound radioactivity was counted by a TopCount Microplate Scintillation Counter (Packard Instrument Company).

The preferred compounds show a Ki value (μM) in mouse on TAAR1 in the range of <0.2 μM. Values for representative compounds are shown in the table below.

| | Ki (μM) |
|---|---|
| Example | mouse |
| 7 | 0.0273 |
| 8 | 0.0017 |
| 8a | 0.0031 |
| 9 | 0.0171 |
| 11 | 0.0009 |
| 11a | 0.0058 |
| 12 | 0.1594 |
| 12a | 0.1751 |
| 13 | 0.0133 |
| 13a | 0.0527 |
| 14 | 0.0007 |
| 14a | 0.0018 |
| 15 | 0.0006 |
| 15a | 0.008 |
| 17 | 0.0003 |

The present invention also provides pharmaceutical compositions containing compounds of the invention, for example, compounds of formula I or pharmaceutically acceptable salts thereof and a pharmaceutically acceptable carrier. Such pharmaceutical compositions can be in the form of tablets, coated tablets, dragées, hard and soft gelatin capsules, solutions, emulsions or suspensions. The pharmaceutical compositions also can be in the form of suppositories or injectable solutions.

The pharmaceutical compositions of the invention, in addition to one or more compounds of the invention, contain a pharmaceutically acceptable carrier. Suitable pharmaceutically acceptable carriers include pharmaceutically inert, inorganic or organic carriers. Lactose, corn starch or derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are however usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Pharmaceutical compositions containing a compound of formula I or a pharmaceutically acceptable salt thereof and a therapeutically inert carrier are also an object of the present invention, as is a process for their production, which comprises bringing one or more compounds of formula I and/or pharmaceutically acceptable acid addition salts and, if desired, one or more other therapeutically valuable substances into a galenical administration form together with one or more therapeutically inert carriers.

The most preferred indications in accordance with the present invention are those, which include disorders of the central nervous system, for example the treatment or prevention of depression, psychosis, Parkinson's disease, anxiety and attention deficit hyperactivity disorder (ADHD).

The dosage at which compounds of the invention can be administered can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage can be administered as single dose or in divided doses and, in addition, the upper limit can also be exceeded when this is found to be indicated.

Tablet Formulation (Wet Granulation)

| | | mg/tablet | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

### Manufacturing Procedure

1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

Capsule Formulation

| | | mg/capsule | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

### Manufacturing Procedure

1. Mix items 1, 2 and 3 in a suitable mixer for 30 minutes.
2. Add items 4 and 5 and mix for 3 minutes.
3. Fill into a suitable capsule.

### EXPERIMENTAL

The following examples illustrate the invention but are not intended to limit its scope.

### Example 1

### (R)-5,5-Dimethyl-4-phenyl-4,5-dihydro-oxazol-2-ylamine

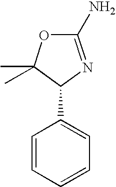

A solution of (R)-1-amino-2-methyl-1-phenyl-propan-2-ol (2.40 g) in THF (20 ml) was treated under an argon atmosphere with K₂CO₃ (2.41 g) and a solution of cyanogen bromide (1.85 g) in THF (20 ml). The reaction mixture was stirred at r.t. overnight, then filtered. The residue was washed with THF. The filtrate was concentrated. The crude product was purified by column chromatography (silica gel: Isolute® Flash-NH₂ from Separtis; gradient: CH₂Cl₂−>CH₂Cl₂/MeOH 9:1) to give the title compound (0.98 g) as off-white solid. MS (ISP): 191.4 ([M+H]⁺)

### Example 2

### 4-Cyclohexyl-5-methyl-4,5-dihydro-oxazol-2-ylamine

### a) 1-(3,4-Dichloro-phenyl)-propane-1,2-dione-1-oxime

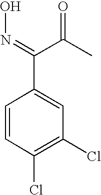

A solution of 3,4-dichlorophenylacetone (5.0 g) in AcOH (7 ml) was cooled to 15° C. and treated dropwise with a solution of sodium nitrite (2.0 g) in AcOH (7 ml). The reaction mixture was maintained at 15° C. for 30 min and then allowed to reach r.t. Water (10 ml) and dioxane (10 ml) were added, and stirring was continued overnight. The reaction mixture was evaporated. The residue was crystallized from EtOH (8 ml) and water (8 ml). The crude product was purified by column chromatography (silica gel; gradient: CH₂Cl₂−>CH₂Cl₂/MeOH 9:1) to give the title compound (1.15 g) as white solid. MS (ISP): 230.0 ([M+H]⁺)

### b) 1-Amino-1-cyclohexyl-propan-2-ol

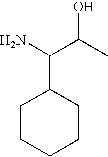

A solution of 1-(3,4-dichloro-phenyl)-propane-1,2-dione 1-oxime (0.5 g) in EtOH (30 ml) was hydrogenated at 2.5 bar at r.t. overnight in the presence of PtO₂ (50 mg). The catalyst was filtered off and washed with EtOH, the filtrate was concentrated. The crude product was purified by column chromatography (silica gel: Isolute® Flash-NH₂ from Separtis; gradient: CH₂Cl₂−>CH₂Cl₂/MeOH 9:1) to give the title compound (218 mg; CAS 90726-26-4) as colorless liquid. MS (ISP): 158.2 ([M+H]⁺⁾

### c) 4-Cyclohexyl-5-methyl-4,5-dihydro-oxazol-2-ylamine

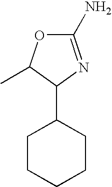

The title compound was prepared in analogy to example 1 from 1-amino-1-cyclohexyl-propan-2-ol. White solid. MS (ISP): 183.3 ([M+H]⁺)

### Example 3

### (4R,5S)-5-Ethyl-4-phenyl-4,5-dihydro-oxazol-2-ylamine

### a) 1-Phenyl-butane-1,2-dione 1-oxime

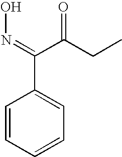

A solution of benzyl ethyl ketone (5.0 g) in EtOH (50 ml) was treated dropwise at 0° C. under an argon atmosphere with tert-butyl nitrite (4.3 ml). After 5 min, a solution of NaOEt (2.8 g) in EtOH (50 ml) was added dropwise. The reaction mixture was stirred at r.t. overnight, then concentrated. The residue was dissolved in water and extracted with EtOAc. The organic layers were dried over MgSO₄, filtered and evaporated. The crude product was purified by column chromatography (silica gel; gradient: CH₂Cl₂−>CH₂Cl₂/MeOH 19:1) to give the title compound (4.04 g) as yellow solid. MS (ISP): 178.3 ([M+H]⁺⁾

### b) 1-Amino-1-phenyl-butan-2-ol

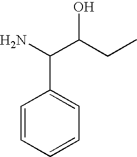

A solution of 1-phenyl-butane-1,2-dione 1-oxime (1.60 g) in EtOH (50 ml) was hydrogenated at 130 bar in the presence of Raney nickel (480 mg; Degussa 46.5%) at 70° C. After cooling, the reaction mixture was filtered. The catalyst was washed with EtOH. The filtrated was concentrated. The crude product was purified by column chromatography (silica gel: Isolute® Flash-NH₂ from Separtis; gradient: CH₂Cl₂−>CH₂Cl₂/MeOH 9:1) to give the title compound (1.26 g) as yellow solid. MS (ISP): 166.4 ([M+H]⁺)

### c) 5-Ethyl-4-phenyl-4,5-dihydro-oxazol-2-ylamine

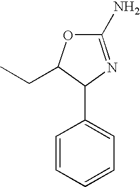

The title compound was prepared in analogy to example 1 from 1-amino-1-phenyl-butan-2-ol. White solid. MS (ISP): 191.4 ([M+H]⁺)

### d) (4R,5S)-5-Ethyl-4-phenyl-4,5-dihydro-oxazol-2-ylamine

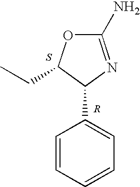

5-Ethyl-4-phenyl-4,5-dihydro-oxazol-2-ylamine (814 mg) was separated by chiral HPLC (Chiralpak AD, EtOH/heptane=10:90) to yield the title compound (310 mg) as white solid. MS (ISP): 191.4 ([M+H]⁺)

### Example 4

### (4R,5S)-5-Methyl-4-phenyl-4,5-dihydro-oxazol-2-ylamine

### a) (1RS,2SR)-1-Amino-1-phenyl-propan-2-ol

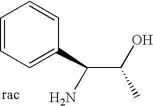

A solution of (2RS,3RS)-2-methyl-3-phenyl-oxirane (2.10 g; CAS 23355-97-7), lithium perchlorate (3.41 g) and 25% aqueous NH₃-solution (70 ml) in THF (30 ml) were placed in an autoclave and heated to 125° C. (9 bar) for 16 hrs. After cooling, the mixture was taken up in water and CH₂Cl₂. The aqueous layer was extracted twice with CH₂Cl₂. The combined organic layers were dried over MgSO₄, filtered and concentrated. The crude product was purified by column chromatography (silica gel: Isolute® Flash-NH₂ from Separtis; gradient: cyclohexane −>CH₂Cl₂−>CH₂Cl₂/MeOH 9:1) to give the title compound (1.77 g) as white solid. MS (ISP): 152.2 ([M+H]⁺)

### c) (4RS,5SR)-5-Methyl-4-phenyl-4,5-dihydro-oxazol-2-ylamine

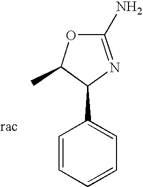

The title compound was prepared in analogy to example 1 from (1RS,2SR)-1-amino-1-phenyl-propan-2-ol. White solid. MS (ISP): 177.3 ([M+H]⁺)

### d) (4S,5R)-5-Methyl-4-phenyl-4,5-dihydro-oxazol-2-olamine

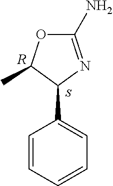

(4RS,5SR)-5-Methyl-4-phenyl-4,5-dihydro-oxazol-2-ylamine (1.7 g) was separated by chiral HPLC (Chiralpak AD, EtOH/heptane=10:90) to yield the title compound (453 mg) as white solid. MS (ISP): 177.4 ([M+H]⁺)

### Example 5

### (4RS,5RS)-5-Methyl-4-phenyl-4,5-dihydro-oxazol-2-ylamine

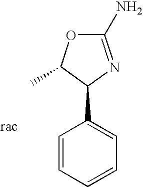

To a stirred solution of trans-(3-methyl styrene (1.00 g) at r.t. in acetonitrile (15 ml) under an argon atmosphere were added silver cyanate (1.39 g) and EtOAc (20 ml). The mixture was cooled in an ice bath and a solution of I₂ (2.58 g) in EtOAc (30 ml) (difficult dissolution!) was added dropwise for 15 min. The ice bath was removed and stirring at r.t. was continued overnight. The mixture was filtered and the cake was washed with EtOAc. The filtrate was concentrated to leave a dark syrup which was taken up in 25% aqueous NH₃-solution (sticky paste, impossible to stir). The mixture was heated to 85° C. and then stirred for 5 hrs. The mixture was cooled to r.t. and extracted with CH₂Cl₂. The combined organics were washed with brine (30 ml), dried over MgSO₄, filtered and concentrated. The crude product was purified by column chromatography (silica gel: Isolute® Flash-NH₂ from Separtis; gradient: cyclohexane −>CH₂Cl₂−>CH₂Cl₂/MeOH 9:1). The product fractions were combined and concentrated. The residue was triturated with a mixture of cyclohexane (5 ml) and CH₂Cl₂ (0.5 ml). The product was collected by filtration and washed with cyclohexane. The title compound (188 mg) was obtained as off-white solid. MS (ISP): 177.4 ([M+H]⁺)

### Example 6

### (4R,5R)-5-Methyl-4-phenethyl-4,5-dihydro-oxazol-2-ylamine

### a) ((R)-2-Oxo-1-phenethyl-propyl)-carbamic acid tert-butyl ester

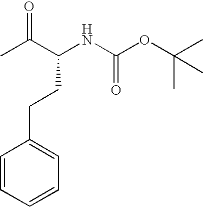

To a stirred, cooled (0° C.) solution of [(R)-1-(methoxy-methyl-carbamoyl)-3-phenyl-propyl]-carbamic acid tert-butyl ester (4.06 g; CAS 171357-71-4) in THF (30 ml) under an argon atmosphere was added dropwise 3M MeMgC1 in THF (16.8 ml) over 10 min. The ice bath was removed and the clear brown solution was stirred at r.t. for 1 hr. The mixture was cooled in an ice bath and carefully treated with 30 ml of 2 N HCl (strong bubbling!). Then it was extracted with EtOAc. The combined organics were washed with brine (30 ml), dried over MgSO₄, filtered and concentrated to leave the title compound as a light brown viscous oil. MS (ISP): 300.4 ([M+Na]⁺).

The crude product was used in the next reaction step without further purification.

### b) ((R)-2-Hydroxy-1-phenethyl-propyl)-carbamic acid tert-butyl ester

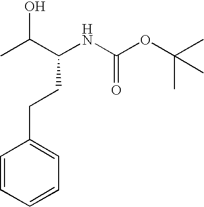

To a stirred solution of ((R)-2-oxo-1-phenethyl-propyl)-carbamic acid tert-butyl ester at 0° C. in ethanol (50 ml) under an argon atmosphere was added carefully NaBH₄ (827 mg). The ice bath was removed and stirring at r.t. was continued for 4 h. The clear colorless solution was concentrated to leave a white sticky solid which was taken up in CH₂Cl₂ (50 ml) and 1 N NaOH (50 ml). The aqueous phase was back extracted with CH₂Cl₂ (50 ml). The combined organics were washed with brine, dried over MgSO₄, filtered and concentrated to leave the title compound (2.89 g) as 3:1 mixture of epimers. White solid. MS (ISP): 302.3 ([M+Na]⁺) The crude product was used in the next reaction step without further purification.

### c) (R)-3-Amino-5-phenyl-pentan-2-ol

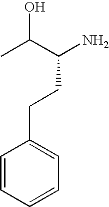

To a stirred solution of ((R)-2-hydroxy-1-phenethyl-propyl)-carbamic acid tert-butyl ester (2.88 g) at r.t. in dioxane (20 ml) under an argon atmosphere was added 4 M HCl solution in dioxane (25.8 ml). Stirring at r.t. was then continued for 4 hrs. The mixture (clear colorless solution) was concentrated to leave a light yellow gum. The residue was taken up in 1 N HCl (50 ml) and washed with EtOAc (50 ml). The aqueous phase was brought to pH 12 by the addition of 4 N NaOH. The product was extracted with EtOAc. The combined organics were washed with brine, dried over MgSO₄, filtered and concentrated to leave the title compound (1.69 g) as a 3:1 mixture of epimers. Light yellow viscous oil. MS (ISP): 180.2 ([M+H]⁺).

The crude product was used in the next reaction step without further purification.

### d) (2R,3R)-Amino-5-phenyl-pentan-2-ol

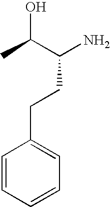

(R)-3-Amino-5-phenyl-pentan-2-ol (1.69 g) was separated by chiral HPLC (Chiralpak AD, EtOH/heptane=15:85) to yield the first eluting (2R,3R)-amino-5-phenyl-pentan-2-ol (318 mg) as viscous, light yellow oil. MS (ISP): 180.2 ([M+H]⁺).

The second eluting epimer (2S,3R)-amino-5-phenyl-pentan-2-ol (857 mg) was isolated as viscous, light yellow oil. MS (ISP): 180.2 ([M+H]⁺).

### e) (4R,5R)-5-Methyl-4-phenethyl-4,5-dihydro-oxazol-2-ylamine

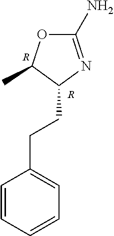

The title compound was prepared in analogy to example 1 from (2R,3R)-amino-5-phenyl-pentan-2-ol. Off-white solid. MS (ISP): 205.3 ([M+H]⁺).

### Example 7

### (4R,5S)-5-Methyl-4-phenethyl-4,5-dihydro-oxazol-2-ylamine

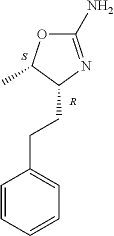

The title compound was prepared in analogy to example 1 from (2S,3R)-amino-5-phenyl-pentan-2-ol (example 6.d). White solid. MS (ISP): 205.3 ([M+H]⁺).

### Example 8

### (4S,5S)-5-Methyl-4-phenethyl-4,5-dihydro-oxazol-2-ylamine and (4S,5R)-5-methyl-4-phenethyl-4,5-dihydro-oxazol-2-ylamine

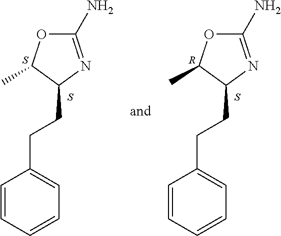

The title compounds were prepared in analogy to example 6/7 from [(S)-1-(methoxy-methyl-carbamoyl)-3-phenyl-propyl]-carbamic acid tert-butyl ester (CAS 183444-03-3). (4S,5S)-5-Methyl-4-phenethyl-4,5-dihydro-oxazol-2-ylamine: off-white solid. MS (ISP): 205.3 ([M+H]⁺). (4S,5R)-5-Methyl-4-phenethyl-4,5-dihydro-oxazol-2-ylamine: white solid.MS (ISP): 205.3 ([M+H]⁺).

### Example 9

### (S)-5-Ethyl-4-phenethyl-4,5-dihydro-oxazol-2-ylamine

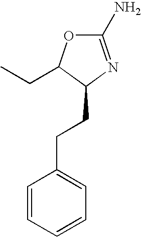

The title compound was prepared in analogy to example 6 starting from [(S)-1-(methoxy-methyl-carbamoyl)-3-phenyl-propyl]-carbamic acid tert-butyl ester (CAS 183444-03-3) and ethylmagnesium chloride. Colorless oil. MS (ISP): 219.4 ([M+H]⁺).

### Example 10

### (4S,5R)-4-Phenethyl-5-trifluoromethyl-4,5-dihydro-oxazol-2-ylamine

### a) (2R,3S)-3-Amino-1,1,1-trifluoro-5-phenyl-pentan-2-ol

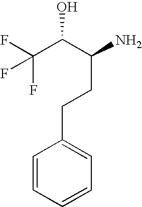

To a stirred solution of (2S,3S)-3-dibenzylamino-1,1,1-trifluoro-5-phenyl-pentan-2-ol (134 mg; Eur. J. Org. Chem. 2004, 1558-1566), at r.t. in MeOH (5 ml) under an argon atmosphere was added Pd(OH)₂ (20% Pd on C; 14 mg). The black suspension was stirred at r.t. under a hydrogen atmosphere overnight. The catalyst was filtered off and the cake was washed with methanol. The filtrate was concentrated to leave a light yellow solid. The crude product was isolated by column chromatography (silica gel: Isolute® Flash-NH₂ from Separtis; gradient: cyclohexane −>CH₂Cl₂−>CH₂Cl₂/MeOH 9:1) to give the title compound (47 mg; CAS 402733-46-4) as viscous brown oil. MS (ISP): 234.2 ([M+H]⁺).

### b) (4S,5R)-4-Phenethyl-5-trifluoromethyl-4,5-dihydro-oxazol-2-ylamine

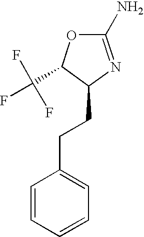

The title compound was prepared in analogy to example 1 from (2S,3S)-3-amino-1,1,1-trifluoro-5-phenyl-pentan-2-ol. Colorless viscous oil. MS (ISP): 259.2 ([M+H]⁺).

### Example 11

### (4S,5R)-5-Methyl-4-(3-phenyl-propyl)-4,5-dihydro-oxazol-2-ylamine and (4S,5S)-5-methyl-4-(3-phenyl-propyl)-4,5-dihydro-oxazol-2-ylamine

### a) [(S)-1-(Methoxy-methyl-carbamoyl)-4-phenyl-butyl]-carbamic acid tert-butyl ester

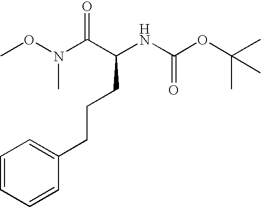

To a stirred, cooled (−15 to −20° C.) solution of (S)-2-tert-butoxycarbonylamino-5-phenyl-pentanoic acid (8.17 g; CAS 98628-27-4) in dichloromethane under an argon atmosphere were added N-methyl morpholine (3.4 ml) and N,O-dimethylhydroxylamine hydrochloride (2.99 g). N-(3-Dimethylaminopropyl)-N′-ethylcarbodiimide hydrochloride (EDCI; 5.87 g) was then added portionwise over a period of 5 min and stirring at −15° C. to −20° C. was continued for 1 h. CH₂Cl₂ (65 ml) was added to the mixture. The layers were separated and the aqueous phase was back extracted with CH₂Cl₂ (65 ml). The combined organics were washed with brine (65 ml), dried over MgSO₄, filtered and concentrated to leave the title compound (8.20 g) as an orange viscous oil.

The crude product was used in the next reaction step without further purification.

### b) (4S,5R)-5-Methyl-4-(3-phenyl-propyl)-4,5-dihydro-oxazol-2-ylamine and (4S,5S)-5-methyl-4-(3-phenyl-propyl)-4,5-dihydro-oxazol-2-ylamine

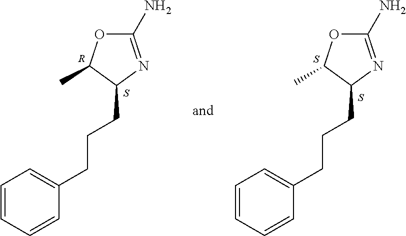

The title compounds were prepared in analogy to example 6/7 from [(S)-1-(methoxy-methyl-carbamoyl)-4-phenyl-butyl]-carbamic acid tert-butyl ester. (4S,5R)-5-Methyl-4-(3-phenyl-propyl)-4,5-dihydro-oxazol-2-ylamine: light yellow solid. MS (ISP): 219.1 ([M+H]⁺). (4S,55)-5-Methyl-4-(3-phenyl-propyl)-4,5-dihydro-oxazol-2-ylamine: colorless viscous oil. MS (ISP): 219.1 ([M+H]⁺).

### Example 12

### (4S,5S)-4-Benzyl-5-methyl-4,5-dihydro-oxazol-2-ylamine and (4S,5R)-4-benzyl-5-methyl-4,5-dihydro-oxazol-2-ylamine

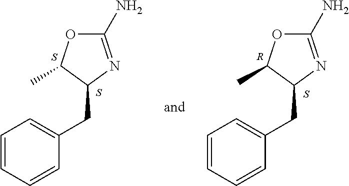

The title compounds were prepared in analogy to example 6/7 from [(S)-1-(methoxy-methyl-carbamoyl)-2-phenyl-ethyl]-carbamic acid tert-butyl ester (CAS 87694-53-9). (4S,5S)-4-Benzyl-5-methyl-4,5-dihydro-oxazol-2-ylamine: colorless viscous solid. MS (ISP): 191.4 ([M+H]⁺). (4 S,5R)-4-Benzyl-5-methyl-4,5-dihydro-oxazol-2-ylamine: off-white solid. MS (ISP): 191.4 ([M+H]⁺).

### Example 13

### (4S,5S)-4-(2-Cyclohexyl-ethyl)-5-methyl-4,5-dihydro-oxazol-2-ylamine

### a) [S)-3-Cyclohexyl-1-(methoxy-methyl-carbamoyl)-propyl]-carbamic acid tert-butyl ester

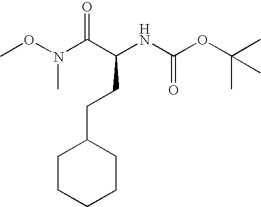

To a stirred, cooled (0° C.) solution of (S)-2-tert-butoxycarbonylamino-4-cyclohexyl-butyric acid (5.73 g; CAS 143415-51-4) in dichloromethane (75 ml) under an argon atmosphere were added N-methyl morpholine (2.43 ml) and N,O-dimethylhydroxylamine hydrochloride (2.15 g). To the reaction mixture was then added portionwise over a period of 5 min N-(3-dimethylaminopropyl)-N′-ethylcarbodiimide hydrochloride (4.23 g) and stirring at 0° C. was continued for 3 h 30. Aqueous 1 N HCl (75 ml) was added, followed by CH₂Cl₂ (75 ml). The layers were separated, and the aqueous phase was back extracted with CH₂Cl₂ (75 ml). The combined organics were washed with brine, dried over MgSO₄, filtered and concentrated to leave [(S)-3-cyclohexyl-1-(methoxy-methyl-carbamoyl)-propyl]-carbamic acid tert-butyl ester (6.30 g) as a light yellow viscous oil. MS (ISP): 351.4 ([M+Na]⁺)

### b) [(S)-1-(2-Cyclohexyl-ethyl)-2-oxo-propyl]-carbamic acid tert-butyl ester

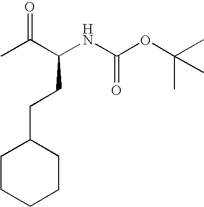

To a stirred, cooled (0° C.) solution of [(S)-3-cyclohexyl-1-(methoxy-methyl-carbamoyl)-propyl]-carbamic acid tert-butyl ester (6.30 g) in THF under an argon atmosphere was added dropwise over 10 min a solution of methyl magnesium chloride (3 M in THF; 25.6 ml). The ice bath was removed and the clear brown solution was stirred at r.t. for 1 h 30. The mixture was cooled in an ice bath and carefully treated with 60 ml of 2 N HCl (60 ml; strong bubbling!), then extracted with EtOAc. The aqueous phase was back extracted with EtOAc. The combined organics were washed with brine, dried (MgSO₄), filtered and concentrated to leave [(S)-1-(2-cyclohexyl-ethyl)-2-oxo-propyl]-carbamic acid tert-butyl ester (5.23 g) as a light yellow viscous oil. MS (ISP): 284.2 ([M+H]⁺)

### c) [([(S)-1-(2-Cyclohexyl-ethyl)-2-hydroxy-propyl]-carbamic acid tert-butyl ester

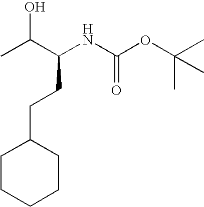

To a stirred solution of [(S)-1-(2-cyclohexyl-ethyl)-2-oxo-propyl]-carbamic acid tert-butyl ester (5.22 g) at 0° C. in ethanol (75 ml) under an argon atmosphere was added carefully NaBH₄ (1.39 g). The ice bath was removed and stirring at r.t. was continued overnight. The mixture was concentrated to leave an off-white paste which was taken up in CH₂Cl₂. The insoluble material was filtered off and washed with CH₂Cl₂. The filtrate was concentrated. The crude product was purified by column chromatography (silica gel; gradient: cyclohexane −>cyclohexane/EtOAc 3:7) to give [(S)-1-(2-cyclohexyl-ethyl)-2-hydroxy-propyl]-carbamic acid tert-butyl ester (4.32 g; 3:1 mixture of epimers) as white solid. MS (ISP): 308.2 ([M+Na]⁺)

### d) (S)-3-Amino-5-cyclohexyl-pentan-2-ol

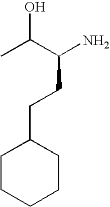

To a stirred solution of [(S)-1-(2-cyclohexyl-ethyl)-2-hydroxy-propyl]-carbamic acid tert-butyl ester (4.32 g) at r.t. in dioxane (37 ml) under an argon atmosphere was added 4 M HCl solution in dioxane (37.8 ml). The mixture was stirred at r.t. overnight and concentrated to leave an off-white sticky solid which was dissolved in 50 ml H₂O. The pH of the solution was brought to ˜1 by the addition of 3 N HCl. It was washed with EtOAC (50 ml). The aqueous phase was brought to pH>12 by the addition of 4 N NaOH. The resulting white slurry was extracted with EtOAc. The combined organics were washed with brine, dried over MgSO₄, filtered and concentrated to leave (S)-3-amino-5-cyclohexyl-pentan-2-ol (2.76 g; 3:1 mixture of epimers) as a light yellow viscous oil. MS (ISP): 186.3 ([M+H]⁺)

### e) (4S,5S)-4-(2-Cyclohexyl-ethyl)-5-methyl-4,5-dihydro-oxazol-2-ylamine and (4S,5R)-4-(2-cyclohexyl-ethyl)-5-methyl-4,5-dihydro-oxazol-2-ylamine

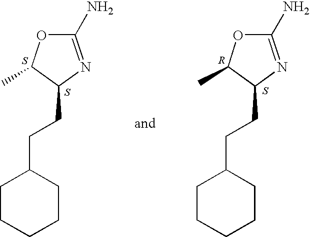

In analogy to example 1(S)-3-amino-5-cyclohexyl-pentan-2-ol was converted to (S)-4-(2-cyclohexyl-ethyl)-5-methyl-4,5-dihydro-oxazol-2-ylamine. Epimers were separated by chromatography (silica gel: Isolute® Flash-NH₂ from Separtis; gradient: CH₂Cl₂−>CH₂Cl₂/MeOH 9:1) to give (4S,5S)-4-(2-cyclohexyl-ethyl)-5-methyl-4,5-dihydro-oxazol-2-ylamine (42 mg) as colorless viscous oil, MS (ISP): 211.1 ([M+H]⁺), and (4S,5R)-4-(2-cyclohexyl-ethyl)-5-methyl-4,5-dihydro-oxazol-2-ylamine (215 mg) as white solid, MS (ISP): 211.1 ([M+H]⁺).

### Example 14

### (4S,5R)-4-[2-(4-Chloro-phenyl)-ethyl]-5-methyl-4,5-dihydro-oxazol-2-ylamine and (4S,5S)-4-[2-(4-chloro-phenyl)-ethyl]-5-methyl-4,5-dihydro-oxazol-2-ylamine

### a) (2S,5R)-2-[2-(4-Chloro-phenyl)-ethyl]-5-isopropyl-3,6-dimethoxy-2,5-dihydro-pyrazine

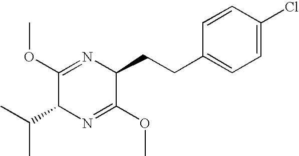

To a stirred, cooled (−78° C.) solution of (2R)-(−)-2,5-dihydro-3,6-dimethoxy-2-isopropylpyrazine (3.22 g) in THF (25 ml) under an argon atmosphere was added dropwise n-buthyl lithium 1.6 M in hexane (12.0 ml). The temperature was kept below −70° C. during the addition. When addition was complete, the mixture was stirred at −78° C. for 1 h, then a solution of 4-chlorophenethyl bromide (5.14 g) in THF (45 ml) was added dropwise. The reaction mixture was stirred overnight, slowly warming up to r.t. Then, the mixture was diluted with Et₂O (70 ml) and sat. aq. NH₄Cl (70 ml). The aqueous phase was extracted with Et₂O. The combined organics were washed with brine, dried over MgSO₄, filtered and concentrated. The crude product was purified by column chromatography (silica gel; gradient: cyclohexane −>cyclohexane/EtOAc 4:1) to give the title compound (3.09 g) as light yellow viscous oil. MS (ISP): 323.2 ([M+H]⁺)

### b) (S)-2-Amino-4-(4-chloro-phenyl)-butyric acid methyl ester

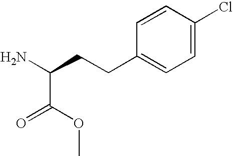

To a stirred solution of (2S,5R)-2-[2-(4-chloro-phenyl)-ethyl]-5-isopropyl-3,6-dimethoxy-2,5-dihydro-pyrazine (3.09 g) at r.t. in acetonitrile (36 ml) under an argon atmosphere were added H₂O (12 ml) and TFA (4.4 ml). The mixture was then stirred overnight at r.t. The pH was brought to ˜11 by the addition of 10% Na₂CO₃. The mixture was extracted with EtOAc. The combined organics were washed with brine, dried over MgSO₄, filtered and concentrated. The crude product was purified by column chromatography (silica gel; gradient: cyclohexane −>EtOAc −>EtOAc/MeOH 85:15) to give the title compound (1.86 g) as viscous light yellow oil. MS (ISP): 228.2 ([M+H]⁺)

### c) (S)-2-tert-Butoxycarbonylamino-4-(4-chloro-phenyl)-butyric acid methyl ester

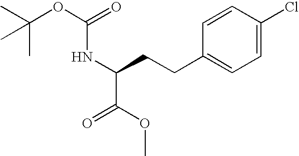

To a stirred solution of (S)-2-amino-4-(4-chloro-phenyl)-butyric acid methyl ester (1.85 g) at r.t. in acetonitrile (20 ml) under an argon atmosphere were added triethylamine (1.2 ml), NaHCO₃ (1.03 g) and Boc₂O (1.95 g). The suspension was stirred at r.t. for overnight. The solids were removed by filtration and the filtrate was concentrated. The residue was taken up in EtOAc, washed with 1 N HCl, H₂O and brine, dried over MgSO₄, filtered and concentrated. The crude product was purified by column chromatography (silica gel; gradient: cyclohexane −>cyclohexane/EtOAc 3:1) to give the title compound (2.35 g) as light yellow viscous oil. MS (ISP): 328.3 ([M+H]⁺)

### d) (S)-2-tert-Butoxycarbonylamino-4-(4-chloro-phenyl)-butyric acid

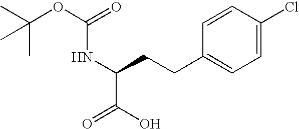

To a stirred solution of (S)-2-tert-butoxycarbonylamino-4-(4-chloro-phenyl)-butyric acid methyl ester at r.t. (2.34 g) in methanol (10 ml) under an argon atmosphere was added 1 N NaOH (10.3 ml). The suspension was stirred at r.t. for 3 h, soon turning again to a clear light yellow solution. The methanol was distilled off. The aqueous residue was brought to pH ˜1 by the addition of 5 N HCl. The product was extracted with EtOAc. The combined organics were washed with brine, dried over MgSO₄, filtered and concentrated. The resulting gum was triturated in a mixture of n-heptane (20 ml) and EtOAc (2 ml). The suspension was stirred at r.t. for 30 min. The solid was collected by filtration, washed with n-heptane and dried to give the title compound (1.29 g) as white solid. MS (ISN): 312.1 ([M−H]⁻)

### e) (4S,5R)-4-[2-4-Chloro-phenyl)-ethyl]5-dihydro-oxazol-2-ylamine and (4S,5S)-4-[2-(4-chloro-phenyl)-ethyl]-5-methyl-4,5-dihydro-oxazol-2-ylamine

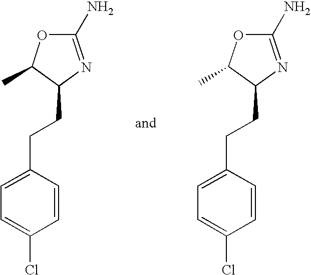

In analogy to example 11 (S)-2-tert-Butoxycarbonylamino-4-(4-chloro-phenyl)-butyric acid was converted to the title compounds.

(4S,5R)-4-[2-(4-Chloro-phenyl)-ethyl]-5-methyl-4,5-dihydro-oxazol-2-ylamine: off-white solid. MS (ISN): 239.0 ([M+H]⁺).

(4S,5S)-4-[2-(4-Chloro-phenyl)-ethyl]-5-methyl-4,5-dihydro-oxazol-2-ylamine: viscous colorless oil. MS (ISN): 239.0 ([M+H]⁺).

### Example 15

### (4S,5R)-4-[2-(3,4-Dichloro-phenyl)-ethyl]-5-methyl-4,5-dihydro-oxazol-2-ylamine and (4S,5S)-4-[2-(3,4-dichloro-phenyl)-ethyl]-5-methyl-4,5-dihydro-oxazol-2-ylamine

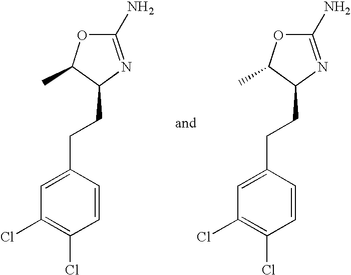

The title compounds were prepared in analogy to example 14, using 4-(2-bromo-ethyl)-1,2-dichloro-benzene as alkylating agent in the first step.

(4S,5R)-4-[2-(3,4-Dichloro-phenyl)-ethyl]-5-methyl-4,5-dihydro-oxazol-2-ylamine: off-white solid. MS (ISP): 273.2 ([M+H]⁺).

(4S,5S)-4-[2-(3,4-Dichloro-phenyl)-ethyl]-5-methyl-4,5-dihydro-oxazol-2-ylamine: light-brown viscous solid. MS (ISP): 273.2 ([M+H]⁺).

### Example 16

### (4S,5R)-5-Ethyl-4-(3-phenyl-propyl)-4,5-dihydro-oxazol-2-ylamine and (4S,5S)-5-ethyl-4-(3-phenyl-propyl)-4,5-dihydro-oxazol-2-ylamine

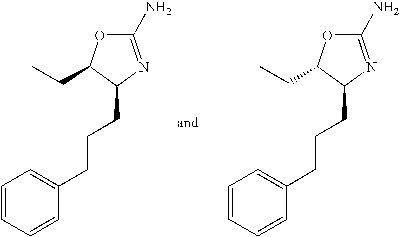

The title compounds were prepared in analogy to example 6/7, starting from [(S)-1-(methoxy-methyl-carbamoyl)-4-phenyl-butyl]-carbamic acid tert-butyl ester (example 11.a) and ethylmagnesium chloride.

(4S,5R)-5-Ethyl-4-(3-phenyl-propyl)-4,5-dihydro-oxazol-2-ylamine: white solid. MS (ISP): 233.2 ([M+H]⁺).

(4S,55)-5-Ethyl-4-(3-phenyl-propyl)-4,5-dihydro-oxazol-2-ylamine: orange viscous solid. MS (ISP): 233.2 ([M+H]⁺).

### Example 17

### (4S,5R)-4-[3-(4-Chloro-phenyl)-propyl]-5-methyl-4,5-dihydro-oxazol-2-ylamine

### a) (S)-2-tert-butoxycarbonylamino-5-(4-chloro-phenyl)-pentanoic acid

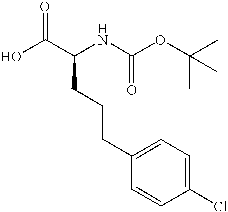

The title compound was prepared in analogy to example 14a-d starting from 1-chloro-4-(3-iodo-propyl)-benzene (CAS 90562-26-8) in place of 4-chlorophenethyl bromide. (S)-2-tert-butoxycarbonylamino-5-(4-chloro-phenyl)-pentanoic acid: light yellow viscous oil. MS (ISP): 326.1 ([M−H]⁻).

### b) (4S,5R)-4-[3-(4-Chloro-phenyl)-propyl]-5-methyl-4,5-dihydro-oxazol-2-ylamine

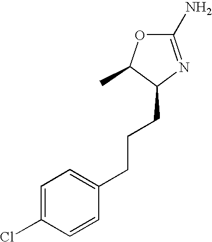

The title compound was prepared in analogy to example 11 starting from (S)-2-tert-butoxycarbonylamino-5-(4-chloro-phenyl)-pentanoic acid in place of (S)-2-tert-butoxycarbonylamino-5-phenyl-pentanoic acid.

(4S,5R)-4-[3-(4-Chloro-phenyl)-propyl]-5-methyl-4,5-dihydro-oxazol-2-ylamine: off-white solid. MS (ISP): 253.1 ([M+H]⁺).

1. A compound of formula I 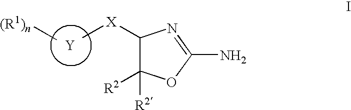
wherein
R¹ is halogen;
R² is lower alkyl or lower alkyl substituted by halogen;
R^{2′} is hydrogen, lower alkyl or lower alkyl substituted by halogen;
X is a bond, —CH₂—, —CH₂CH₂— or —CH₂CH₂CH₂—;
Y is phenyl or cyclohexyl; and
n is 0, 1 or 2;
or a pharmaceutically suitable acid addition salt thereof.

2. The compound of claim 1, having formula IA, IB, IC or ID, 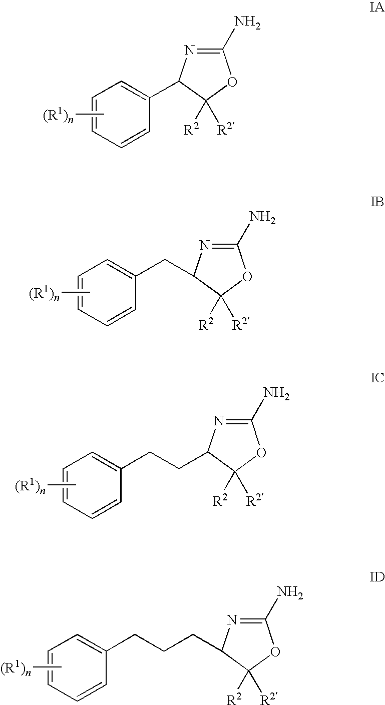
wherein
R¹ is halogen;
R² is lower alkyl or lower alkyl substituted by halogen;
R²′ is hydrogen, lower alkyl or lower alkyl substituted by halogen; and
n is 0, 1 or 2;
or a pharmaceutically suitable acid addition salt thereof.

3. The compound of formula IB according to claim 2, selected from the group consisting of
(4S,5S)-4-benzyl-5-methyl-4,5-dihydro-oxazol-2-ylamine and
(4S,5R)-4-benzyl-5-methyl-4,5-dihydro-oxazol-2-ylamine.

4. The compound of formula IC according to claim 2, selected from the group consisting of
(4R,5S)-5-methyl-4-phenethyl-4,5-dihydro-oxazol-2-ylamine;
(4S,5S)-5-methyl-4-phenethyl-4,5-dihydro-oxazol-2-ylamine;
(4S,5R)-5-methyl-4-phenethyl-4,5-dihydro-oxazol-2-ylamine;
(S)-5-ethyl-4-phenethyl-4,5-dihydro-oxazol-2-ylamine;
(4S,5R)-4-[2-(4-chloro-phenyl)-ethyl]-5-methyl-4,5-dihydro-oxazol-2-ylamine;
(4S,5S)-4-[2-(4-chloro-phenyl)-ethyl]-5-methyl-4,5-dihydro-oxazol-2-ylamine;
(4S,5R)-4-[2-(3,4-dichloro-phenyl)-ethyl]-5-methyl-4,5-dihydro-oxazol-2-ylamine; and
(4S,5S)-4-[2-(3,4-dichloro-phenyl)-ethyl]-5-methyl-4,5-dihydro-oxazol-2-ylamine.

5. The compound of formula ID according to claim 2, selected from the group consisting of
(4S,5R)-5-methyl-4-(3-phenyl-propyl)-4,5-dihydro-oxazol-2-ylamine;
(4S,5S)-5-methyl-4-(3-phenyl-propyl)-4,5-dihydro-oxazol-2-ylamine; and
(4S,5R)-4-[3-(4-chloro-phenyl)-propyl]-5-methyl-4,5-dihydro-oxazol-2-ylamine.

6. The compound of claim 1, having formula IE or IF 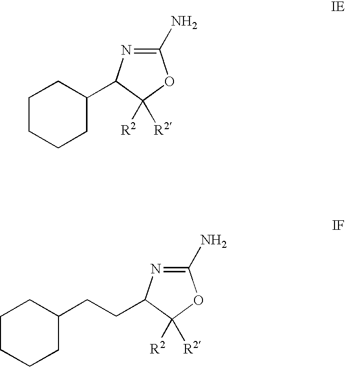
wherein
R² is lower alkyl or lower alkyl substituted by halogen; and
R² is hydrogen, lower alkyl or lower alkyl substituted by halogen;
or a pharmaceutically suitable acid addition salt thereof.

7. The compound of formula IE according to claim 6, wherein the compound is
(4-cyclohexyl-5-methyl-4,5-dihydro-oxazol-2-ylamine.

8. The compound of formula IF according to claim 6, selected from the group consisting of
(4S,5S)-4-(2-cyclohexyl-ethyl)-5-methyl-4,5-dihydro-oxazol-2-ylamine and
(4S,5R)-4-(2-cyclohexyl-ethyl)-5-methyl-4,5-dihydro-oxazol-2-ylamine.

9. A pharmaceutical composition comprising a therapeutically effective amount of a compound of formula I 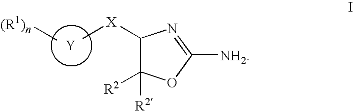
wherein
R¹ is halogen;
R² is lower alkyl or lower alkyl substituted by halogen;
R² is hydrogen, lower alkyl or lower alkyl substituted by halogen;
X is a bond, —CH₂—, —CH₂CH₂— or —CH₂CH₂CH₂—;
Y is phenyl or cyclohexyl; and
n is 0, 1 or 2;
or a pharmaceutically suitable acid addition salt thereof and a pharmaceutically acceptable carrier.
